(19) United States
(12) Patent Application Publication
(10) Pub. No.: US 2013/0004563 A1
(43) Pub. Date: Jan. 3, 2013

(54) MULTIPARTICULATE S-ADENOSYLMETHIONINE COMPOSITIONS AND RELATED METHODS

(76) Inventors: SYED SHAH, Delray Beach, FL (US); NOREEN HASSAN, BOCA RATON, FL (US)

(21) Appl. No.: 13/489,936

(22) Filed: Jun. 6, 2012

Related U.S. Application Data
(60) Provisional application No. 61/494,036, filed on Jun. 7, 2011.

(51) Int. Cl. A61K 9/14; A61P 25/24; A61P 19/08; B05D 5/00; A61P 25/00; A61P 1/16; A61K 9/48; A61K 31/7076; A61P 19/02

(52) U.S. Cl. 424/451; 424/494; 514/46; 424/495; 427/2.21

(57) ABSTRACT

Multiparticulate compositions having S-adenosylmethionine an active ingredient are disclosed. The multiparticulates have spheroidal core comprising S-adenosylmethionine, microcrystalline cellulose, and hydroxypropyl methylcellulose; a sub-coat comprising hydroxypropyl methyl cellulose on the spheroidal core; and an enteric coat on the sub-coated spheroidal core. The average diameter of the particulates is about 0.1-3 mm. Other aspects of the invention include methods of making and methods of using the multiparticulate compositions.

### CROSS-REFERENCE TO RELATED APPLICATION

This application claims priority to U.S. Provisional Patent Application Ser. No. 61/494,036 filed on Jun. 7, 2011 titled “Multiparticulate Compositions Comprising S-adenosylmethionine (SAMe) and Related Methods,” which is incorporated by reference herein in its entirety.

### FIELD OF THE INVENTION

The invention relates to multiparticulate compositions comprising the active ingredient S-adenosylmethionine (SAMe), and more particularly, to controlled release multiparticulate compositions comprising SAMe.

### BACKGROUND

S-adenosyl-L-methionine, hereinafter referred to as “SAMe”, is an endogenous molecule that plays an important role in cellular metabolism. SAMe is a precursor of glutathione, an antioxidant naturally produced by the liver.

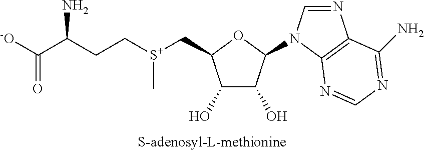

Because SAMe is relatively unstable in vitro, it is typically associated with a relatively large sized anion when used in pharmaceutical products (Morana 2000). Examples of SAMe salts that are stable enough to be used in pharmaceutical products include the tosylate disulfate, butanedisulfonate, di-para-toluene sulfonate disulfate, tri-para-toluene sulfonic acid salts and the like. Examples of some stable salts of SAMe are described in U.S. Pat. Nos. 3,954,726 and 4,057,686.

SAMe stability can be enhanced by avoiding a low pH environment and by associating it with trehalose in the lyophilized state (Morana, 2002). The stabilization in the lyophilized state is attributed to the glassy state, which locks in the mobility of this molecule. While trehalose is acceptable for parenteral dosage forms, it is not preferred for oral dosage forms because it may cause flatulence when administered orally.

Pharmaceutical compositions of SAMe are used for the treatment of various disorders including liver disease, depression (Mischoulon 2002), and osteoarthritis (Gregory 2008). Unfortunately, some of the current techniques for administering SAMe to patients suffer from the fact that SAMe can cause side effects such as stomach pain, diarrhea, nausea and flatulence. Some immediate release forms of SAMe may cause gastrointestinal upsets and/or allow SAMe to degrade during product storage (Gregory 2008).

SAMe bioavailability is relatively low with a level of 0.5% to 1.0% for immediate release formulations and higher for enteric-coated single unit tablets (Yang 2009). In the body, SAMe has a relatively short half-life of approximately 6 hours for single dose and about 4 to 5 hours for multiple dose administrations (Yang 2009). Thus, frequent dosing of SAMe is often required. Typically, SAMe is administered as single unit, immediate release, or enteric-coated tablets, in doses between 400 to 1600 mg, divided into 2 to 3 divided doses per day. But, since SAMe has a relatively short half life in the body, delivery of SAMe via immediate release tablets or delayed release tablets is not ideal because it may cause wide variations of SAMe concentration in systemic circulation.

Less frequent dosing and longer release times can be achieved by administering SAMe in a sustained release formula. Still, however, the sustained release formulas currently on the market typically require twice daily dosing, release the SAMe locally to a small section of the gastrointestinal tract, and have the potential to sit for long periods in the stomach before actually entering the upper intestinal tract. Accordingly, the effectiveness of a single unit dosage form that is enteric-coated and is non-disintegrating is liable to a higher risk of dose dumping (EMEA-CPMP Guidance 1999). The lack of timely transit of the enteric-coated tablet through the pyloric sphincter can lead to a failure-to-treat and dose dumping can result in increased local adverse effects.

### SUMMARY

In view of the foregoing, it is an object of the invention to provide a controlled-release SAMe composition that has been adapted to minimize the side-effects of SAMe in patients and to provide for an extended release of SAMe in the intestines, which improves SAMe bioavailablity and dosage effectiveness.

In a composition aspect of the invention, a SAMe composition comprises a plurality of independently dispersible particulates, each independently dispersible particulate comprising: a spheroidal core comprising about 70%-90% w/w S-adenosylmethionine, about 15%-25% w/w microcrystalline cellulose, and about 0.5%-1.5% w/w hydroxypropyl methylcellulose; a sub-coat on the spheroidal core, the subcoat comprising hydroxypropyl methyl cellulose present in an amount of about 2%-4% w/w of the independently dispersible particulates; and an enteric coat on the sub-coated spheroidal core, the enteric coat being about 5%-15% w/w of the independently dispersible particulates; wherein the average diameter of the independently dispersible particulates is about 0.1-3 mm.

The enteric coat may be selected from methacrylic acid co-polymer, cellulose acetate phthalate, polyvinyl acetate phthalate, or a combination thereof. Alternatively, the enteric coat may comprise a polymeric material that forms a film around the core and a pore former material that generates pores in the film under intestinal pH conditions. In a particular embodiment, the polymeric material is ethyl cellulose and the pore former material is sodium alginate.

In some embodiments, the composition further comprises a S-adenosylmethionine permeation enhancer adapted to assist S-adenosylmethionine in permeating biological tissue. In a particular embodiment, the S-adenosylmethionine permeation enhancer is a p-glycoprotein efflux pump inhibitor such as, for example, polysorbate 80.

In some embodiments, the core further comprises a pellet, wherein the S-adenosylmethionine is located on an outer surface of the pellet. The pellet may be a non-pareil pellet or microcrystalline cellulose pellet, for example.

The composition is preferably present in a pharmaceutically acceptable dosage form for being administered to a patient.

In a method of use aspect of the invention, a method of treating a physiological condition in a patient comprises administering the composition of the invention to the patient. In a preferred embodiment, the physiological condition is selected from depression, fibromyalgia, osteoarthritis, headache, liver malfunction, or a combination thereof. Administering the composition to the patient may comprise administering a capsule having the independently dispersible particulates therein, combining the composition with an acidic food vehicle, or providing a blend of the composition and an acidic food vehicle to the patient through a feeding tube.

In a method of making aspect of the invention, a method of making a controlled-release multiparticulate composition of S-adenosylmethionine comprises: producing a spheroidal core comprising about 70%-90% w/w S-adenosylmethionine, about 15%-25% w/w microcrystalline cellulose, and about 0.5%-1.5% w/w hydroxypropyl methylcellulose; coating the spheroidal core with a sub-coat comprising hydroxypropropyl methyl cellulose, the sub-coat being about 2%-4% w/w of the particulates in the multiparticulate composition; applying an enteric coat to the sub-coated spheroidal core, the enteric coat being about 5%-15% w/w of the particulates in the multiparticulate composition; and wherein the average diameter of particulates in the multiparticulate composition is about 0.1-3 mm.

In some embodiments, the spheroidal core is produced by extrusion and spheronization. In a particular example, the spheroidal core is produced by blending the S-adenosylmethionine, microcrystalline cellulose, and hydroxypropyl methylcellulose with water to form a met mass and extruding the wet mass, cutting the extruded wet mass into pieces, spheronizing the pieces, and drying the spheronized pieces. The spheronized pieces are preferably dried at a temperature of about 50° C.-60° C.

In some embodiments, the spheroidal core is produced by coating a non-pareil or microcrystalline cellulose pellet with the S-adenosylmethionine, microcrystalline cellulose, and hydroxypropyl methylcellulose.

These and other objects, aspects, and advantages of the present invention will be better appreciated in view of the following detailed description of preferred embodiments.

### DETAILED DESCRIPTION OF PREFERRED EMBODIMENTS

In the Summary above and in the Detailed Description of Preferred Embodiments, reference is made to particular features (including method steps) of the invention. It is to be understood that the disclosure of the invention in this specification includes all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will be thorough and complete, and will convey the scope of the invention to those skilled in the art.

One aspect of the invention is to provide multiparticulate compositions comprising SAMe for treating physiological disorders related to a reduction of SAMe in the body of a patient. Examples of physiological disorders that may be treated with such a multiparticulate composition include depression, fibromyalgia, osteoarthritis, headache, and/or liver malfunction. The multiparticulate compositions of the invention advantageously permit the particulates in the composition to pass to the intestines without substantially releasing SAMe in the stomach, thus preventing the undesirable side effects or reduced efficacy of SAMe that may result otherwise.

Further, the composition provides a reduced release in the stomach and an elevated release at a substantially neutral pH, such as the pH found in the intestines. As used herein, a substantially neutral pH environment means an environment having a pH of about 7, including, but not limited to a pH of between about 6.5 to about 7.5, also including the pH environment of the intestines.

The multiparticulate compositions of the invention provide an advantageous SAMe non-parenteral delivery vehicle that can be administered to a patient. A multiparticulate composition of the invention comprises a plurality of independently dispersible particulates that are preferably spheroidal in shape and are preferably configured for incorporation into a capsule, sachet, or packet-type oral delivery dosage form. By being independently dispersible, the particulates are able to disperse within the intestines and to individually release SAMe therefrom. Each particulate is preferably sized to fit through the pyloric sphincter in a relaxed state. The diameter of the particulates is preferably in the range of about 0.1-3 mm or, more preferably, about 1-2.5 mm.

In certain embodiments, the particulates comprise a spheroidal core with an enteric coat over the core. The particulates may also have an optional sub-coat between the core and enteric coat. In a preferred embodiment, the sub-coat comprises hydroxypropyl methyl cellulose, also known as “HPMC” or “hypromellose.” A suitable HPMC sub-coat is applied to the core as a solution of 10% w/w HPMC in water. The particulates may also include one or more additional coatings such as a sealant coating or a color coating on the enteric coating.

The active ingredient in the core is SAMe. As used herein, the terms “SAMe” and “S-adenosylmethionine” are used interchangeably to mean S-adenosylmethionine and derivatives thereof; and S-adenosylmethionine salts and derivatives thereof. Examples of SAMe salts include, but are not limited to the tosylate disulfate, butanedisulfonate, di-para-toluene sulfonate, disulfate, tri-para-toluene sulfonic acid salts and the like.

The core may also include one or more of a pharmaceutically acceptable filler, stabilizer, binder, surfactant, processing aid, and/or disintegrant. By way of example only, suitable materials for performing some of these functions are provided. A preferred filler is microcrystalline cellulose. A preferred binder is a cellulosic water soluble polymer such as cellulose ether. A preferred processing aid is colloidal silicon dioxide. A preferred disintegrant is croscarmellose sodium.

In order to achieve a controlled-release of SAMe from the core, SAMe is preferably dispersed in a release-controlling polymer. Preferred release-controlling polymers include HPMC, carboxy methyl cellulose, ethyl cellulose, cyclodextran, polyethylene glycol, and poly(propylene oxide).

In a preferred embodiment, the core contains about: 70%-90% w/w S-adenosylmethionine, about 15%-25% w/w filler, and about 0.5%-1.5% w/w release controlling polymer. Here the % w/w is relative to the total weight of the uncoated core.

An enteric coat is applied over the uncoated core or, if the sub-coating is present, over the sub-coat. The enteric coat is preferably applied so that it comprises about 5-15% w/w of the enteric coated particulates. Preferred materials for making the enteric coat include methacrylic acid co-polymer, cellulose acetate phthalate, polyvinyl acetate phthalate, ethyl cellulose, and HPMC. In some embodiments, the enteric coat is a mixture of a polymeric material that forms a film around the core and a pore former material that generates pores in the film under intestinal pH conditions. A preferred example of such a mixture includes ethyl cellulose as the polymeric material and sodium alginate as the pore former.

Examples of suitable methacrylic acid based copolymers include Eudragit L30D-55 or Kollicoat MAE 30 DP. These materials may be combined with other materials such as plasticizers for forming an enteric coating solution. In a preferred embodiment, an enteric coating solution comprises about 50%-80% w/w water, about 0.1-1.5% w/w plasticizer, about 2-8% anti-adherent, and about 10-40% copolymer. By way of example only, a suitable plasticizer is triethyl citrate and a suitable anti-adherent is PlasACRYL T20. It is preferred that the enteric coating material be applied to the core at a temperature of about 40° C.-60° C., or, more preferably about 50° C.

Methods of making the multiparticulate compositions in accordance with another aspect of the invention will now be described. The core may be prepared by wet granulating the core materials into a wet mass, extruding the wet mass to form an extrudate, cutting the extrudate into a plurality of pieces, and spheronizing the pieces. The spheronized core pieces are preferably dried at a temperature of about 50° C.-60° C. The spheronized pieces are subsequently coated with the enteric coating material.

Alternatively, the core may be prepared by coating a non-pareil pellet or microcrystalline cellulose pellet with SAMe dispersed in the release controlling polymer. The pellet may be coated by spray coating, powder layering, or fluidized bed coating, for example.

The enteric coating is typically applied in a fluidized bed coater. The enteric coated particulates are subsequently dried. The dried enteric coated multiparticulates may then be prepared into a suitable pharmaceutical dosage form. A typical preferred dosage form contains about 250 mg of the particulates. Depending on the desired dosage, however, this may be adjusted.

Excipients such as sodium carboxy methyl cellulose (CMC), or disaccharides such as sucrose or lactose, may be used in combination with the binder to prepare the wet mass that is needed for extrusion and spheronization. Alternatively, the CMC or disaccharide may be coated together with SAMe and a glass state stabilizer such as HPMC may be added to the non-pareil seed or MCC pellet coating application. These two methods for preparing multi-particulates mentioned in this section can be employed with SAMe in the presence of a neutral pH buffer such as dibasic sodium or potassium phosphate or di-calcium phosphate, etc. if desired.

The multiparticulate compositions of the invention are preferably formulated to be taken non-parenterally by a patient for treating one or more physiological conditions that can be remediated by SAMe. In a method of use aspect of the invention, a method of treating a physiological condition in a patient comprises administering a composition of the invention to the patient. The term “patient” refers to humans or other animals considered as having one or more physiological conditions that can be remediated with SAMe. Examples of such physiological conditions include depression, fibromyalgia, osteoarthritis, headache, and liver malfunction. The term “administering” refers to the giving or applying of a substance. In a preferred embodiment, administering the composition to the patient includes administering a capsule having the independently dispersible particulates therein.

In another preferred embodiment, administering the composition to the patient includes combining the independently dispersible particulates with an acidic food vehicle, such as an acidic, semi-solid food or drink. This administration technique may be particularly useful with patients who have difficulty swallowing. In such embodiments, the particulates are preferably loaded into a sachet that the patient or a caregiver can easily open for sprinkling the particulates onto the acidic food vehicle. When the patient ingests the acidic food vehicle, the patient also ingests the particulates. Preferred acidic food vehicles include food products like applesauce, fruit slurries, fruit juices, or the like.

In such embodiments, a multiparticulate composition of the invention may be administered through a feeding tube, such as a nasogastric feeding tube, gastric feeding tube, jejunostomy feeding tube, or the like. In general, a feeding tube is a medical device that medical caregivers use to deliver nutrition to a patient who has difficulty swallowing or is unable to swallow. A nasogastric feeding tube is a feeding tube that is passed through the nostrils and esophagous to the stomach. A gastric feeding tube is a feeding tube that that is inserted into the stomach via an incision in the patient's abdomen. A jejunostomy feeding tube is a feeding tube that has been surgically inserted into the jejunum of the small intestine. In practice, the multiparticulate composition is blended with the acidic food vehicle and the blend is administered to the patient by passing the blend through the feeding tube.

Doses of the multiparticulate composition may be administered sporadically when needed or may be administered as part of a long term treatment.

An effective dose is a quantity sufficient to affect a condition in the subject, such as affecting a medical condition or process in the body of the subject. In some preferred embodiments, effective doses of SAMe are between about 100 to 2000 mg.

These embodiments of the invention have many advantages. Some but not all of those advantages are listed here. Not all of the advantages are required by all embodiments of the invention.

One advantage of the multiparticulate compositions of the invention is that the they will provide a more reliable release of SAMe when compared to single-unit sustained release formulations that are presently available, without concern for dosing of the patient under the fed or fasted state. They will further provide a prolonged exposure to the SAMe both locally and systemically as compared to the single-unit sustained release formulations. The use of multiparticulate formulations of the present invention comprising SAMe may allow for less frequent dosing and may also allow for dosing with a lower total amount of SAMe. Dispersion of the particulates in the lumen of the small bowel, prior to release of the SAMe, may reduce the incidence of side effects seen with the other SAMe formulations. Further, single unit sustained release formulations tend to release the SAMe only in the local vicinity of the dosage form. The multiparticulate compositions of the present invention can avoid this problem because the particulates will disperse in the intestinal tract to provide a delocalized dose of SAMe therein.

### EXAMPLE

### Preparation of a Preferred Multiparticulate Composition

This section describes a prophetic example of a preferred multiparticulate composition of the invention and its method of making. The example is not intended to limit the scope of the invention in any way.

Equipment.

The equipment that may be used to create the compositions herein include the following: top loading balances, hand screens (12, 14, 16, 18, Pan, 70 mesh), Rotap sieve shaker, IKA mixer, KitchenAid food processor (pre-milling), Hobart mixer, LCI Benchtop Granulator, Fitz mill equipped with a 0.065″ screen, Jet Mill, Key International high sheer mixer, Glatt GPCC-3 fluid bed drier, Glatt GPCC-3 fluid bed dried with 7″ Wurster, Karl Fischer moisture analyzer, and a spheronizer.

SAMe Core Formation.

The core is prepared utilizing the following steps and settings. 955 grams SAMe, 226.8 grams Microcrystalline Cellulose (Avicel Ph 102; FMC Corporation), and 11.94 grams Methocel A15 LV (Dow) are low shear granulated in a 0.5 Gallon (2 Liter) Hobart or other granulation mixer and mixed at low speed for about 5 minutes. USP water is sprayed into the mixer to achieve peak granulation moisture, and this is blended for about an additional 10-30 minutes to form a wet mass.

The wet mass is extruded through a 0.6, 0.8, or 1.0 mm-hole perforated metal screen using a LCI Benchtop Granulator at speed setting 10.

The extrudate is spheronized in 25-30 grams sub lots using a Caleva Model 120 spheronizer equipped with a small pyramid plate at high speed for 1-5 minutes.

The combined spheronization sub lots are dried in a GPCG-3 or similar fluid bed dryer for about 45-75 minutes with an inlet temperature set point between about 50° C. and 60° C. and a process air flow of about 40-60 cfm.

The finished dried SAMe multiparticulates are collected between 12-mesh and 20-mesh screens.

Sub-Coat.

1000 grams of spheronized SAMe cores are placed into a Glatt GPCG-3 fluid bed drier and the sub-coating is sprayed onto the cores in the form of a 10% hypromellose (hypromellose E5) aqueous solution that is maintained at about room temperature.

The sub-coating solution (306 g USP Water (T>55° C.) and 34 g hypromellose E5 (Dow) is applied to the cores using the following parameters: the inlet temperature is maintained at about 50° C.; the air flow is maintained at about 50 cfm; the spray rate is maintained between about 6.0 and 11.0 g/min; and the filter shake cycle is about 45/3 seconds (Time Between Shaking/Shaking Time). The fluid bed drier is setup with a 1.0 mm Schlick 970 nozzle port, and 2×360 air cap setting, a 1.5 cm partition setting, and a multiparticulate bottom plate or equivalent.

Enteric Coat.

The enteric coat is applied to the cores in a fluidized bed coater (7″ wurster) as a liquid solution. The formula for the enteric coating is 1160 grams USP Water (RT), 506.6 grams BASF Kollicoat MAE 30 DP, 75.7 grams PlasACRYL T20 (Colorcon), and 7.9 grams Triethyl Citrate USP, which is mixed a minimum of 20 minutes and screen through a 40-mesh screen prior to use.

The enteric coating solution is applied to 1000 grams of SAMe dried spheronized cores using the following parameters: the inlet temperature is maintained at about 50° C.; the air flow is maintained at about 50 cfm; the spray rate is maintained between 11.0 and 15.0 g/min; the atomization air pressure is maintained at about 3.0 bar; and the filter shake cycle is 45/3 seconds (Time Between Shaking/Shaking Time). The fluid bed drier is set up with a 1.0 mm Schlick 970 nozzle port, and 2×360 air cap setting, a 1.5 cm partition setting, and a multiparticulate bottom plate or equivalent.

A finish coat may be applied over the enteric coating, and is applied in a same or similar manner as the enteric coating.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described. The skilled should understand that the methods and materials used and described are examples and may not be the only ones suitable for use in the invention.

Any publications, patent applications, patents, and other references mentioned herein are incorporated by reference in their entirety as if they were part of this specification. However, in case of conflict, the present specification, including any definitions, will control.

In the specification set forth above there have been disclosed typical preferred embodiments of the invention, and although specific terms are employed, the terms are used in a descriptive sense only and not for purposes of limitation. The invention has been described in some detail, but it will be apparent that various modifications and changes can be made within the spirit and scope of the invention as described in the foregoing specification and as defined in the appended claims.

### REFERENCES CITED

Morana, A. 2000 “Synthesis and characterization of a new class of a stable S-adenosyl-L-methionine” Inst. Of Food Science and Technology: 2000 Jan. 20; 194(1):61-68.
Mischoulon, D and Fava, Maurizio 2002 “Role of S-adenosyl-L-methionine in the treatment of depression: a review of the evidence” American Journal of Clinical Nutrition: November 2002; Vol. 76, No. 5, 1158S-1161S.
Gregory, P. et. al. 2008 “Dietary Supplements for Osteoarthritis” American Family Physician, Jan. 15, 2008: Vol. 77, No. 2; 177-184.
Yang, J et. al. 2009 “Pharmacokinetic Properties of S-Adenosylmethionine After Oral and Intravenous Administration of Its Tosylate Disulfate Salt: A Multiple-Dose, Open-Label, Parallel-Group Study in Healthy Chinese Volunteers” Clinical Therapeutics: 2009; Vol. 31, No. 2: 311-320.
Morana, A “Stabilization of S-Adenosyl-L-methionine promoted by trehalose” Biochimica et Biophysica Acta 2002; 1573: 105-108.
EMEA (European Agency for the Evaluation of Medicinal Products) CPMP (Committee for Proprietary Medicinal Products) (London, 29 Jul. 1999) “Note for Guidance on Quality of Modified Release Products; A: Oral Dosage Forms.” 6-7

That which is claimed is:

1. A composition comprising a plurality of independently dispersible particulates, each independently dispersible particulate comprising:
a spheroidal core comprising about 70%-90% w/w S-adenosylmethionine, about 15%-25% w/w microcrystalline cellulose, and about 0.5%-1.5% w/w hydroxypropyl methylcellulose;
a sub-coat on the spheroidal core, the subcoat comprising hydroxypropyl methyl cellulose present in an amount of about 2%-4% w/w of the independently dispersible particulates; and
an enteric coat on the sub-coated spheroidal core, the enteric coat being about 5%-15% w/w of the independently dispersible particulates;
wherein the average diameter of the independently dispersible particulates is about 0.1-3 mm.

2. The composition of claim 1, wherein the enteric coat is selected from methacrylic acid co-polymer, cellulose acetate phthalate, polyvinyl acetate phthalate, or a combination thereof.

3. The composition of claim 1, wherein the enteric coat comprises a polymeric material that forms a film around the core and a pore former material that generates pores in the film under intestinal pH conditions.

4. The composition of claim 3, wherein the polymeric material is ethyl cellulose and the pore former material is sodium alginate.

5. The composition of claim 1, further comprising a S-adenosylmethionine permeation enhancer adapted to assist S-adenosylmethionine in permeating biological tissue.

6. The composition of claim 5, wherein the S-adenosylmethionine permeation enhancer is a p-glycoprotein efflux pump inhibitor.

7. The composition of claim 6, wherein the p-glycoprotein efflux pump inhibitor is polysorbate 80.

8. The composition of claim 1, wherein the core further comprises a pellet and wherein the S-adenosylmethionine is located on an outer surface of the pellet.

9. The composition of claim 8, wherein the pellet is a non-pareil or microcrystalline cellulose pellet.

10. The composition of claim 1, wherein the multiparticulate composition is present in a pharmaceutically acceptable dosage form.

11. A method of treating a physiological condition in a patient, the method comprising administering the composition of claim 1 to the patient.

12. The method of claim 11, wherein the physiological condition is selected from depression, fibromyalgia, osteoarthritis, headache, liver malfunction, or a combination thereof.

13. The method of claim 11, wherein administering the composition of claim 1 to the patient comprises administering a capsule having the independently dispersible particulates therein.

14. The method of claim 11, wherein administering the composition of claim 1 to the patient comprises combining the composition of claim 1 with an acidic food vehicle.

15. The method of claim 11, wherein administering the composition of claim 1 to the patient comprises providing a blend of the composition and an acidic food vehicle to the patient through a feeding tube.

16. A method of making a controlled-release multiparticulate composition of S-adenosylmethionine, the method comprising:
producing a spheroidal core comprising about 70%-90% w/w S-adenosylmethionine, about 15%-25% w/w microcrystalline cellulose, and about 0.5%-1.5% w/w hydroxypropyl methylcellulose;
coating the spheroidal core with a sub-coat comprising hydroxypropropyl methyl cellulose, the sub-coat being about 2%-4% w/w of the particulates in the multiparticulate composition;
applying an enteric coat to the sub-coated spheroidal core, the enteric coat being about 5%-15% w/w of the particulates in the multiparticulate composition; and
wherein the average diameter of particulates in the multiparticulate composition is about 0.1-3 mm.

17. The method of claim 16, wherein the spheroidal core is produced by extrusion and spheronization.

18. The method of claim 16, wherein the spheroidal core is produced by blending the S-adenosylmethionine, microcrystalline cellulose, and hydroxypropyl methylcellulose with water to form a met mass, extruding the wet mass, cutting the extruded wet mass into pieces, spheronizing the pieces, and drying the spheronized pieces.

19. The method of claim 18, wherein the spheronized pieces are dried at a temperature of about 50° C.-60° C.

20. The method of claim 16, wherein the spheroidal core is produced by coating a non-pareil or microcrystalline cellulose pellet with the S-adenosylmethionine, microcrystalline cellulose, and hydroxypropyl methylcellulose.
